(19) United States
(12) Patent Application Publication
(10) Pub. No.: US 2020/0086272 A1
(43) Pub. Date: Mar. 19, 2020

(54) REMOVAL OF NUCLEIC ACIDS AND FRAGMENTS THEREOF FROM A BIOMASS MATERIAL

(71) Applicant: UNIBIO A/S, Odense (DK)

(72) Inventors: Eleni Ntokou, Odense (DK); Subir Kumar Nandy, Kgs. Lyngby (DK)

(21) Appl. No.: 16/471,310

(22) PCT Filed: Dec. 20, 2017
(86) PCT No.: PCT/EP2017/083683

§ 371 Date: Jun. 19, 2019

(30) Foreign Application Priority Data
Dec. 22, 2016 (DK) PA 2016 71029

(51) Int. Cl. B01D 61/14; A23J 3/20; A23K 10/16; A23L 33/195; B01D 61/58; C12N 1/08; B01D 15/08

(52) U.S. Cl. CPC B01D 61/142; A23J 3/20; A23K 10/16; A23L 33/195; B01D 61/58; B01D 2311/2688; B01D 15/08; B01D 61/145; B01D 61/147; B01D 2311/04; C12N 1/08

(57) ABSTRACT

A method for providing a SCP product from a biomass material is shown wherein the SCP product includes a reduced amount of nucleic acids relative to the naturally occurring amount of nucleic acids in the biomass material, the method including: (i) providing the biomass material; (ii) subjecting the biomass material to a process of cell disruption, providing a disrupted biomass material; (iii) applying the disrupted biomass material to a first separation process resulting in a first retentate comprising proteins and/or cell debris, and a first permeate comprising nucleic acids; (iv) subjecting the first permeate to a second treatment separating the nucleic acids from vitamins, minerals and/or amino acids; (v) optionally, combining the first retentate obtained in step (iii) with the vitamins, minerals and/or amino acids obtained in step (iv), providing the SCP product comprising a reduced amount of nucleic acids relative to the naturally occurring amount of nucleic acids.

### TECHNICAL FIELD OF THE INVENTION

The present invention relates to a method for providing one or more isolates from a biomass. In particular, the present invention relates to an industrial method for removing nucleic acids from a biomass, e.g. for producing single cell protein from cultivated methanotrophic bacteria with a reduced nucleic acid content.

### BACKGROUND OF THE INVENTION

One of the greatest challenges of humankind is how we can feed a fast-growing global population with nutritious and affordable food. The global population is expected to surpass 9 billion by 2050 and according to Food and Agriculture Organization of the United Nations (FAO), they will consume twice as much animal protein by 2050 compared to today. The current anthropogenic pressure on earth's finite resources and the concomitant dynamics of climate change, generate serious concerns about the resilience of the contemporary agricultural feed/food chains have forced the search for alternative protein sources that can replace conventional animal protein sources. Hence, the focus has shifted to exploit microbes as food sources for consumption and in particular products such as SCP has shown to be very interesting. The term “single cell protein” (SCP) was coined in 1968 at a meeting held at the Massachusetts Institute of Technology (MIT) to replace the less aesthetic “microbial protein” and “petroprotein” which were the terms originally used. At present, SCP derived from bacterial biomass is mainly used for animal feed, and in some cases for human consumption, and is expected to be of greater importance in the future.

The term single cell protein (SCP) commonly refers to a proteinaceous product isolated from single celled microorganisms. The proteinaceous product may be in the form of a biomass or a protein extract and comprises cell wall materials of single celled microorganisms from pure or mixed cultures of algae, yeasts, fungi, or bacteria. The single cell protein is traditionally used as an ingredient or a substitute for protein-rich foods, and is suitable for human consumption or as animal feeds.

Utilizing microorganisms to obtain biomass for use in feed and food results in a product that has a higher proportion of nucleic acids than conventional foods. Although the amount of nucleic acids present in SCP varies depending on the specific microorganism employed, generally about 5 to about 18 percent nucleic acids (dry weight) are present in SCP.

RNA, DNA and nucleic acid as such, are not desired in the protein product, such as in the SCP product, as these compounds may have direct or indirect effect on the health of a mammal, such as humans or animals, e.g. by causing gout or gouty arthritis or kidney stones in the mammal.

Conventionally, dietary RNA and DNA are decomposed into nucleic acid fragments in the intestinal lumen, and further decomposed into nucleotides and/or nucleosides and free purine and pyrimidine bases by nucleotide and/or nucleoside phosphatase enzymes in the mucosa.

The metabolism of purine bases results in high levels of uric acid. Humans do not possess the enzyme uricase, which oxidizes uric acid to allantoin, a soluble and excretable metabolite. Consumption of a protein source high in nucleic acids results in hyperuricemia which is defined by abnormally high level of uric acid found in the blood. Uric acid has low solubility at physiological pH values thus forming crystals of uric acid that can be retained in the joints and kidneys, causing gout or gouty arthritis and kidney stones.

Hence, nucleic acids may in excessive or uncontrolled amounts be considered as biogenic substances and are regarded as a limiting factor in the use of SCP derived from algae, yeasts, fungi and bacteria in food products for human nutrition. The normal plasma uric acid concentration in men is 5.1±0.9 mg ml⁻¹ and in women is approximately 1 mg ml⁻¹ less. The Recommended Daily Allowance for protein is 65 grams per day for a 70-kilogram adult male and the Protein Calorie Advisory Group of the United Nations System recommends that the amount of nucleic acid ingested per day from microbial protein should be less than 2 grams with the total nucleic acid from all sources not exceeding a total of 4 grams per day.

There is a variety of methods that have been reported in scientific literature for removal or reduction of the nucleic acid content of SCP. Methods as such enzymatic treatment, acid treatment, base treatment and heat shock have been described. These methods are however, too ineffective and are not able to remove most of the nucleic acids and nucleotide/nucleosides, purines and pyrimidines are still left in the product. Furthermore, the enzymatic or chemical methods may also negatively affect the protein content of the final product which is highly undesirable. Finally, the prior art methods are too complex, require additional processing steps, not applicable in industrial settings, and/or too expensive to be utilized to produce food and feed. Moreover, processes that have been used in the past for nucleic acid removal, such as enzymatic treatment, acid treatment, base treatment and heat shock, affect the SCP product in terms of flavour, odour and colour and since the content of nucleic acid, fragments thereof and nucleotides and/or nucleosides is very high in the traditional SCP product the SCP product, becomes unattractive for food, as it requires to have a mild flavour, odour and colour so as not to influence the palatability of the food or feed.

Hence, there is a need for an improved method for providing isolates from biomasses, such as SCP products where the nucleic acids (e.g. DNA and/or RNA) are removed without affecting flavour, odour and colour of the SCP products or the food product where the SCP products may be used. There is furthermore, a need in the industry for a method which is effective in providing various isolates from biomass and in removing nucleic acids, reducing or without affecting the protein content of the SCP protein, which is simple, reproducible, fast, can handle large volumes, industrially applicable, cheap and/or requires a minimum of handling steps.

### SUMMARY OF THE INVENTION

Thus, an object of the present invention relates to a simplified method for providing one or more isolates, in particular, a SCP product where the nucleic acids (e.g. DNA and/or RNA) are removed without affecting the SCP product.

It is an object of the present invention to provide a method that solves the above-mentioned problems of the prior art with methods resulting in SCP products where flavour, odour and colour are affected, as well as ineffective methods and methods resulting in incapability to remove most of the nucleic acids, adverse effects on the protein content of the final product, too complex methods, requirements of additional processing steps, non-applicability in industrial settings and/or too expensive SCP products to find use in food and feed production.

Thus, one aspect of the invention relates to a method for providing one or more fraction(s) from a biomass material, the method comprises the steps of:
(i) providing the biomass material;
(ii) subjecting the biomass material to a process of cell disruption, providing a disrupted biomass material;
(iii) applying the disrupted biomass material to a first separation process resulting in a first fraction (first retentate) comprising proteins, and a second fraction (first permeate) comprising nucleic acids and vitamins, minerals and/or amino acids;
(iv) subjecting the second fraction (first permeate) to a second treatment resulting in a third fraction (second retentate) comprising nucleic acid and a fourth fraction second permeate) comprising vitamins, minerals and/or amino acids.

Yet an aspect of the present invention relates to a method for providing a Single Cell Protein product (SCP product) from a biomass material wherein said SCP product comprising a reduced amount of nucleic acids relative to the naturally occurring amount of nucleic acid in the biomass material, the method comprising the steps of:
(i) providing the biomass material;
(ii) subjecting the biomass material to a process of cell disruption, providing a disrupted biomass material;
(iii) applying the disrupted biomass material to a first separation process resulting in a first retentate comprising proteins and/or cell debris, and a first permeate comprising nucleic acids and vitamins, minerals and/or amino acids;
(iv) subjecting the first permeate to a second treatment separating the nucleic acids from vitamins, minerals and/or amino acids;
(v) optionally, combining the first retentate obtained in step (iii) with the vitamins, minerals and/or amino acids obtained in step (iv), providing the SCP product comprising a reduced amount of nucleic acids relative to the naturally occurring amount of nucleic acids.

Another aspect of the present invention relates to a method for removing nucleic acids from a biomass material, the method comprises the steps of:
(i) providing the biomass material;
(ii) subjecting the biomass material to a process of cell disruption, providing a disrupted biomass material;
(iii) applying the disrupted biomass material to a first separation process resulting in a first retentate comprising proteins and/or cell debris, and a first permeate comprising nucleic acids and vitamins, minerals and/or amino acids;
(iv) subjecting the first permeate to a second treatment separating the nucleic acids from vitamins, minerals and/or amino acids;
(v) optionally, combining the first retentate obtained in step (iii) with the vitamins, minerals and/or amino acids obtained in step (iv), providing a fraction wherein the nucleic acids have been removed.

Yet another aspect of the present invention relates to a biomass fraction obtainable by a method according to the present invention.

Still another aspect of the present invention relates to a biomass fraction comprising a biomass material and a reduced content of nucleic acids, relative to the naturally occurring amount of nucleic acids in the biomass material.

An even further aspect of the present invention relates to a feed comprising one or more fraction or SCP product according to the present invention.

### BRIEF DESCRIPTION OF THE FIGURES

FIG. 1 shows an embodiment of the present invention for providing a biomass fraction or a SCP product. The method describes a provided biomass material (a cell disrupted biomass material), comprising cell debris, proteins, amino acids (AA), vitamins, minerals, DNA and RNA, obtained after cell disruption. The disrupted biomass material is subjected to the consecutive separation, first by microfiltration (MF) followed by ultrafiltration (UF). The first retentate obtained from the microfiltration comprises the cell debris and the proteins, the first permeate obtained from the microfiltration comprises the vitamins, minerals, amino acids (AA), DNA and RNA. The first permeate is then added to the second separation process, the ultrafiltration process. The second retentate obtained from the ultrafiltration comprises the DNA and RNA, the second permeate obtained from the ultrafiltration comprises the vitamins, minerals, and amino acids (AA). The first retentate and the second permeate are then combined providing the SCP product according to the present invention.

FIG. 2 shows an embodiment of the present invention for providing various fractions from downstream processing of fermented bacterial single cell protein resulting in fractions comprising cell debris, suspended solids, fat, proteins and/or peptides, vitamins/minerals/amino acids and nucleic acids. The fermented single cell protein may preferably be bacterial single cell protein which, in FIG. 2, may be obtained from the fermentation of methanotrophic bacteria in a reactor (1), such as a U-Loop reactor (1). The U-Loop reactor (1) may, during the fermentation, be supplied with methane (1), e.g. provided in the form of biogas, mineral solutions (3), and the needed oxygen (4). During the fermentation process excess CO₂ produced by the methanotrophic bacteria may be discharged from the reactor (1) through the outlet (5). The biomass may be harvested and transferred to the homogenizer (6) which disrupts the cells liberating intracellular proteins and/or peptides, minerals, salts, vitamins etc. From the homogenizer (6) the disrupted biomass is transferred to a decanter (7) where the cell debris fraction (8) may be taken out. The biomass may subsequently be transferred to a clarifier (9) for removing suspended solids (10), like suspended cell debris. The clarified biomass may subsequently be subjected to a fat separator (11) providing a fat fraction (12). The biomass may then be subjected to first separation process (13) comprising either membrane filtration, e.g. by microfiltration (13), or chromatographic separation, e.g. by affinity chromatography (13), providing a protein and/or peptide fraction (14) - a first fraction. The biomass - or a second fraction - is then transferred to a second separation process (15), comprising a membrane filtration, such as an ultrafiltration separation (15) resulting in a fraction (16) comprising the vitamins, minerals and amino acids; and a fraction (17) comprising the nucleic acids.

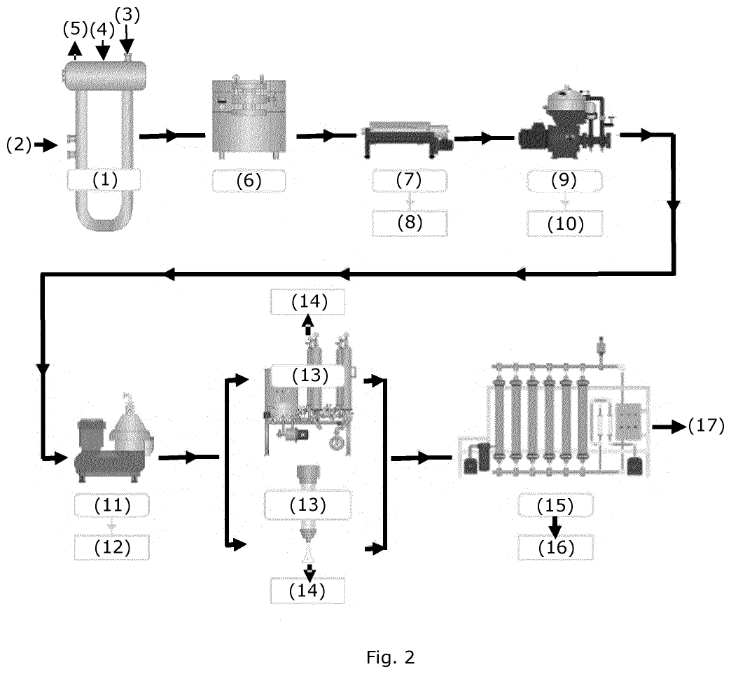

The present invention will now be described in more detail in the following.

### DETAILED DESCRIPTION OF THE INVENTION

Accordingly, there is presently a need in the world for alternative protein sources for feed and food to humans and animals, this need however, will increase dramatically over the coming years as the population continues to increase. Single cell protein (SCP), e.g. obtained from microbial biomass, is a highly potent source as it is cheap, and reproducible.

Microbial protein, such as single cell protein (SCP) requires cultivation of microorganisms in a fermentation tank. Many different fermentation techniques have been described and as an example, the biomass material according to the present invention may be provided by the fermentation process described in WO 2010/069313; WO 2000/70014; or US 2004/0241790, preferably the biomass material is provided by the fermentation process as described in WO 2010/069313, which are all incorporated by reference.

Thus, when the fermentation, e.g. by one of the above-mentioned fermentation methods, has been completed and the biomass material is obtained from the fermenter, the biomass material may be subjected to further downstream processing.

A preferred embodiment of the present invention relates to a method for providing one or more fraction(s) from a biomass material, the method comprises the steps of:

(i) providing the biomass material;

(ii) subjecting the biomass material to a process of cell disruption, providing a disrupted biomass material;

(iii) applying the disrupted biomass material to a first separation process resulting in a first fraction (first retentate) comprising proteins, and a second fraction (first permeate) comprising nucleic acids;

(iv) subjecting the second fraction (first permeate) to a second treatment resulting in a third fraction (second retentate) comprising nucleic acid and a fourth fraction second permeate) comprising vitamins, minerals and/or amino acids.

During fractionation of a biomass material, various valuable fractions individually or in various combinations, may be obtained and used in different applications.

In an embodiment of the present invention the first fraction obtained in step (iii) may be combined with the fourth fraction obtained in step (iv), providing a fifth fraction. In such a combination of fractions a biomass material is provided with reduced amount of nucleic acid.

During processing of the disrupted biomass material provided in step (ii) the viscosity of the disrupted biomass material may be too high complicating pumping and processing of the disrupted biomass material, particularly in the first separation process and/or the second separation process. Thus, in an embodiment of the present invention, the viscosity of the disrupted biomass material may be reduced.

In an embodiment of the present invention the disrupted biomass material provided in step (ii) may be subjected to at least one separation process removing suspended solids from the biomass material. The suspended solids may comprise cells and/or cell debris.

Preferably, the at least one separation process removing suspended solids includes a decanter, a clarifier or a combination hereof. Preferably, the at least one separation process removing suspended solids involves the consecutive treatment of the biomass material of subjecting the disrupted biomass material to decanting and the supernatant from the decanting process is subsequently subjected to clarification. The supernatant obtained from the clarification process may subsequently be subjected to the first separation process as described in step (iii)

The decanting process and/or the clarifying process may mainly remove cells and cell debris from the biomass material, providing a disrupted biomass material with reduced cell debris. The disrupted biomass material obtained from the decanting process (the supernatant obtained from decanting) may constitute a disrupted biomass material with reduced content of cells and cell debris.

In the present context, a disrupted biomass material with reduced content of cells and cell debris comprise less than 10% (w/w) cell or cell debris, such as below 8%, e.g. below 7%, such as below 6%, e.g. below 5%, such as below 4%, e.g. below 3%, such as between 1.5-10%, e.g. between 2-9%, such as between 2.5-8%, e.g. between 3-7%, such as between 3.5-6%, e.g. between 4-5%.

The disrupted biomass material obtained from clarification (the supernatant obtained from clarification) may constitute a disrupted biomass material substantially without cell debris

In the present context, a disrupted biomass material with reduced content of cells and cell debris comprise less than 1.5% (w/w) cell or cell debris, such as below 1%, e.g. below 0.75%, such as below 0.5%, e.g. below 0.25%, such as below 0.1%, e.g. between 0.1-1.5%, e.g. between 0.25-1%, such as between 0.5-0.75%.

The suspended solids, such as cell debris, may be an abundant source of phospholipids. Like vitamins, phospholipids are essential nutrients. They are among the most important substances in the human and animal organism, having a multiple function: as a fat substitute or a source of energy in feed or food products; as physiological agents in metabolism; and as emulsifiers for fats.

In an embodiment of the present invention the disrupted biomass material provided in step (ii) may be subjected to a fat removal process, providing a fat fraction. Preferably, the fat removal process includes a fat separator. Fat removal may be performed before or after decanter and/or clarifier. Even more preferably, the fat removal process is performed after the clarification process.

The fat fraction obtained from the fat removal process mainly consists of fatty acids that may be used for the production of soaps, cosmetics, and industrial mold release agents. The fat fraction may also find use in foodstuffs because they are inexpensive and may add texture and “mouth feel” to processed foods (convenience food).

In a further embodiment of the present invention the first separation process comprises a first membrane filtration or a first chromatographic separation process.

The chromatographic separation process may include a column chromatographic separation process. Preferably, the column chromatographic separation process includes a Packed Bed Chromatography, stirred tank adsorption, Fluidized Bed Chromatography and/or Expanded Bed Chromatography. Preferably, the column chromatographic separation process may be Expanded Bed Chromatography.

Furthermore, the chromatographic separation process may include affinity chromatography, ion exchange chromatography, reversed phase chromatography, hydrophobic interaction chromatography, or a mixture hereof, such as mixed mod chromatography. Preferably, the chromatographic separation process may be affinity chromatography or mixed mode chromatography.

In an embodiment of the present invention, the one or more fraction may be a Single Cell Protein product (a SCP product), a nucleic acid product, a cell/cell debris product, or an amino acid product. In the context of the present invention the terms “SCP product”, “nucleic acid product”, “cell/cell debris product” and “amino acid product” relate to products enriched in SCP, nucleic acid, cell debris or amino acids, respectively.

The nucleic acids isolated according to the present invention may be used, directly or as further processed according to pending regulations, as an ingredient for food or feed. In particular, the nucleic acids isolated according to the present invention may be used as an ingredient for baby food or infant formula.

Another preferred embodiment of the present invention relates to a method for providing a SCP product from a biomass material wherein said SCP product comprising a reduced amount of nucleic acids relative to the naturally occurring amount of nucleic acids in the biomass material, the method comprising the steps of:
(i) providing the biomass material;
(ii) subjecting the biomass material to a process of cell disruption, providing a disrupted biomass material;
(iii) applying the disrupted biomass material to a first separation process resulting in a first retentate comprising proteins and/or cell debris, and a first permeate comprising nucleic acids;
(iv) subjecting the first permeate to a second treatment separating the nucleic acids from vitamins, minerals and/or amino acids;
(v) optionally, combining the first retentate obtained in step (iii) with the vitamins, minerals and/or amino acids obtained in step (iv), providing the SCP product comprising a reduced amount of nucleic acids relative to the naturally occurring amount of nucleic acids.

The biomass material provided in step (i) may be provided from a fermentation tank, preferably from a U-Loop fermenter (preferably as described in WO 010/069313).

As mentioned earlier, large amounts of nucleic acids are produced during the cultivation of microorganisms, and faced with the interest to limit or avoid these risks and disadvantages of having nucleic acids present in the fermentation product, the methods according to the present invention are shown to reduce the content of nucleic acids in the fermentation product by at least 10%, relative to the naturally occurring amount of nucleic acid in the biomass material; such as at least 20%, e.g. at least 30%, such as at least 40%, e.g. at least 50%, such as at least 60%, e.g. at least 70%, such as at least 80%, e.g. at least 90%, such as at least 95%, e.g. at least 98%.

A further preferred embodiment of the present invention relates to a method for removing nucleic acids from a biomass material, the method comprises the steps of:
(i) providing the biomass material;
(ii) subjecting the biomass material to a process of cell disruption, providing a disrupted biomass material;
(iii) applying the disrupted biomass material to a first separation process resulting in a first retentate comprising proteins and/or cell debris, and a first permeate comprising nucleic acids;
(iv) subjecting the first permeate to a second treatment separating the nucleic acids from vitamins, minerals and/or amino acids;
(v) optionally, combining the first retentate obtained in step (iii) with the vitamins, minerals and/or amino acids obtained in step (iv), providing a SCP product wherein the nucleic acids have been removed.

In the present context, the term “nucleic acids” relates to biopolymers, or large biomolecules, essential for all known forms of life. Nucleic acids, include DNA (deoxyribonucleic acid) and RNA (ribonucleic acid) or fragments thereof. Nucleic acids are made from monomers known as nucleotides. In the present context, the terms “nucleotide” and “nucleoside” may be used interchangeably and the simple difference is that nucleosides can be considered nucleotides without a phosphate group.

In the present context, the term “removing nucleic acids” relates to removal of at least 10% of the nucleic acids naturally present in the biomass material (resulting in the fermentation product); such as at least 20%, e.g. at least 30%, such as at least 40%, e.g. at least 50%, such as at least 60%, e.g. at least 70%, such as at least 80%, e.g. at least 90%, such as at least 95%, e.g. at least 98%.

Suitable biocatalysts used in the process and the fermenter according to the invention may preferably be living cells, e.g. microorganisms of natural origin, i.e. wild types, specially selected mutated types or genetically modified types that may be used to produce single cell protein, enriched single cell protein, proteins or peptide extracts, cell extracts, or preparations containing particular beneficial substances to be used for example for food or feed or to be delivered in order to improve or optimize the health, performance or well-being of humans or animals, such as, but not limited to cloven hoofed animals (e.g. cattle, goats, sheep, pigs, etc.), poultry (e.g. fowls, chicken, ducks, goose/geese, turkey, etc.), fish (e.g. salmon, halibut, trout, cod, or other species bred in captivity) or shellfish (e.g. molluscs such as mussels, oysters, shrimps, prawns, lobsters, or scallops).

The biocatalysts are preferably living microorganisms. Fermentation of the microorganisms may be carried out using pure cultures or using blends or a mixture of different microorganisms, e.g. for production of baker's yeast, single cell protein (SCP). The fermentation process may also result in biotransformations (i.e. microbial conversion of different chemicals to other useful chemicals), or production of intracellular or extracellular enzymes, proteins or hormones for use in different industries or in certain products, (e.g. pharmaceuticals, nutraceuticals or compounds for use as diagnostic or analytic agents).

The preferred bacteria for use in the invention are those capable of producing single cell protein, especially a culture comprising methanotrophic bacteria.

In an embodiment of the present invention the biomass material may be a single-cell protein material. Preferably, the single-cell protein material, and the biomass material, comprises a methanotrophic bacteria.

In an embodiment of the present invention the methanotrophic bacteria may optionally be combined with one or more species of other bacteria, e.g. heterotrophic bacteria.

In another embodiment of the present invention, the fermenter may be used for the fermentation of methylotrophic fungi or yeasts such as Pichia stipitis or Pichia pastoris. P. stipitis and P. pastoris are both capable of metabolizing methanol and may be suitable for potential GMO-production.

The preferred methanotrophic bacteria are species of the Methylococcaceae family, especially Methylococcus capsulatus, which utilize methane or methanol as a carbon source and e.g. ammonia, nitrate or molecular nitrogen as a nitrogen source for protein synthesis.

In an embodiment of the present invention the methanotrophic bacteria may be selected from the family Methylococcaceae or the family Methylocystaceae. Preferably, the biomass material comprises a Methylococcus strain. Even more preferably, the Methylococcus strain is Methylococcus capsulatus.

M. capsulatus metabolizes the methane, e.g. from natural gas, into biomass and carbon dioxide. M. capsulatus is also able to metabolize methanol instead of methane. Natural gas frequently contains 5-10% ethane and higher hydrocarbons, and M. capsulatus can only oxidize these hydrocarbons into the corresponding alcohols, aldehydes and carboxylic acids, but cannot oxidize these completely to carbon dioxide and water or utilize them for biomass production.

Accumulated high concentrations of carboxylic acids may inhibit the growth of M. capsulatus. Therefore, it may be useful to co-ferment one or more strains of heterotrophic bacteria with the methanotrophic bacteria for digesting higher hydrocarbons (alcohols, carboxylic acids, etc.) e.g. ethanol, acetate, citrate, etc. or degradation products of partially digested dead or decaying biomass.

Thus, the fermentation broth may, in addition to M. capsulatus, be supplemented with one or more heterotrophic bacteria or yeasts (e.g. Saccharomyces and/or Candida). The co-fermentation is preferably carried out using three heterotrophic bacteria, which are selected for providing a fermentation ecosystem in which all product niches are occupied. Their main function is to exploit acetic acid and other carboxylic acids and degrade them to carbon dioxide, so that carboxylic acid accumulation is avoided.

The following heterotrophic bacteria may be particularly useful to co-ferment with M. capsulatus; Ralstonia sp.; Bacillus brevis; Brevibacillus agri; Alcaligenes acidovorans; Aneurinibacillus danicus and Bacillus firmus. Suitable yeasts may be selected from species of Saccharomyces and/or Candida.

In an embodiment of the present invention, the preferred combination of bacteria may be a co-fermentation of M. capsulatus with Alcaligenes acidovorans (NCIMB 13287), Aneurinibacillus danicus (NCIMB 13288) and Bacillus firmus (NCIMB 13289).

The fermentation broth in the fermenter may preferably continuously be provided with the required amounts of water and nutrient salts, such as ammonium/ammonia, magnesium, calcium, potassium, iron, copper, zinc, manganese, nickel, cobalt and molybdenum in the form of sulphates, chlorides or nitrates, phosphates and pH controlling components, i.e. acids and/or bases, as normally used by the skilled person, e.g. sulphuric acid (H₂SO₄), nitric acid (HNO₃), sodium hydroxide (NaOH), potassium nitrate (KNO₃). The latter is also a suitable nitrogen source for M. capsulatus. The specific details of the fermentation process, and substrates etc. is described in WO 2000/70014 and WO 2010/069313, which are incorporated by reference.

The biomass material produced from fermentation of natural gas will comprise from 60 to 80% by weight crude protein; from 5 to 20% by weight crude fat; from 3 to 12% by weight ash; from 3 to 15% by weight nucleic acids (RNA and DNA).

Hence, in an embodiment of the present invention, the biomass material provided in step (i) may be subjected to a process of cell disruption, providing a disrupted biomass material.

In the present context, the term “cell disruption” relates to a method or process for releasing biological molecules from inside a cell or organism.

In an embodiment of the present invention the process of cell disruption may involve a mechanical or pressurised cell disruption.

In a further embodiment of the present invention the process of cell disruption involves homogenization of the biomass material, subjecting the biomass material to ball milling or shear forces. Preferably, the cell disruption involves homogenization and the homogenization may be a high-pressure homogenization.

In an embodiment of the present invention, the biomass material obtained from the cultivation tank may be subjected to centrifugation and/or filtration process, e.g. an initial ultrafiltration, to remove part of the water present in the biomass material and to form an aqueous paste or slurry prior to homogenization. During such centrifugation the dry-matter content of the biomass material may typically be increased from about 2 to about 15% by weight, e.g. to about 12% by weight. Initial ultrafiltration, may be performed at a temperature between 40 and 50° C., e.g. between 42 and 46° C., and further concentrates the biomass material to a product containing from 10 to 30%, preferably from 15 to 25%, e.g. from 18 to 22% by weight single-cell protein material. The size exclusion used during ultrafiltration will generally be in the range of about 100,000 Daltons.

Following initial ultrafiltration the biomass material may be cooled, preferably to a temperature of from 4-30° C., such as from 10 to 20° C., e.g. to about 15° C., for example by passing the concentrated protein slurry from the ultrafiltration unit over a heat exchanger after which it may be held in a buffer-tank at constant temperature, e.g. for a period of from 1 to 24 hours, preferably 3 to 15 hours, e.g. 5 to 12 hours, at a temperature of from 10 to 20° C., more preferably from 5 to 15° C. at a pH in the range of from 5.5 to 6.5.

Homogenization may be carried out in a conventional high-pressure homogenizer in which the cells may be disrupted by first pressurizing, and then depressurizing the inside of the homogenizer.

Homogenization may preferably be high-pressure homogenization which involves a change in pressure of the biomass material. Preferably change in pressure of the biomass material may be a pressure drop in the range of from 200 to 2,500 bar, such as in the range of 400 to 2,000 bar, e.g. in the range of 600 to 1,500 bar, such as in the range of 1,000 to 1,300 bar, e.g. in the range of 1,200 to 1,250 bar, such as in the range of 1,300 to 2,200 bar, e.g. in the range of 1400 to 2,000 bar, such as above 1,200 bar, e.g. above 1,250 bar, such as above 1,500 bar, e.g. about 2,000 bar.

In an embodiment of the present invention the process of cell disruption provided in step (ii) may be performed under controlled temperature conditions, preferably at a temperature of less than 50° C., particularly preferably from 25 to 50° C., e.g. from 25 to 35° C.

A single step of drop-in pressure may be preferred, however, in an embodiment of the present invention the drop-in pressure may be stepped, such as comprising two or more steps. If two or more steps are provided the drop-in pressure may start with the highest pressure drop and followed by a decrease in successive drops in pressure according to the number of steps.

The homogenization process herein described results in a disrupted biomass material comprising disrupted cells. The disrupted cells may be present in an amount of at least 80% by weight (20% of the cells remain undisrupted), preferably at least 90% by weight, even more preferably at least 95% by weight, even more preferred at least 98% by weight. Typically, the disrupted biomass material may be a relatively viscous protein slurry containing soluble and particulate cellular components, such as proteins; cell debris; RNA; DNA; vitamins; minerals: and amino acids (such as free amino acids).

Thus, in an embodiment of the present invention the disrupted biomass material may be further treated by applying the disrupted biomass material to a first separation process resulting in a first retentate comprising proteins and/or cell debris, and a first permeate comprising nucleic acids.

In an embodiment of the present invention wherein the first separation process may be a first membrane filtration. Preferably, the first membrane filtration may be a microfiltration.

In the present context, the term “microfiltration” (commonly abbreviated to MF) relates to a type of physical filtration process where a contaminated fluid is passed through a special pore-sized membrane to separate microorganisms and suspended particles from process liquid.

In an embodiment of the present invention, the first separation process may have a molecular weight cut-off value (MWCO) of greater than 1,000,000 Dalton, such as greater than 1,200,000 Dalton, e.g. greater than 1,500,000 Dalton.

In a further embodiment of the present invention the first membrane filtration may have a pore size in the range of 0.03-10 μm, such as a pore size in the range of 0.05-5 μm, e.g. a pore size in the range of 0.1-2, such as a pore size in the range of 0.15-1, e.g. a pore size in the range of 0.2-0.75, such as a pore size in the range of 0.25-0.5.

The first membrane filtration may involve an organic polymer membrane, such as polysulfones, poly (styrenes), PVDF (polyvinylidene fluoride) and PAN (polyacrylonitrile) including styrene-containing copolymers such as acrylonitrile-styrene, butadiene-styrene and styrene-vinylbenzylhalide copolymers, polycarbonates, cellulosic polymers, polypropylene, poly (vinyl chloride), poly (ethylene terephthalate); or an inorganic polymer membrane, such as a ceramic membrane filter material. Preferably, the first membrane filtration may involve a ceramic membrane filter material.

In an embodiment of the present invention, the first membrane filtration may be a dynamic disc filter.

In a preferred embodiment of the present invention, the disrupted biomass material may be supplied to a first separation process which involves a microfiltration process, preferably using ceramic membrane filter material. From the microfiltration process a first retentate may be provided, said first retentate comprising proteins and/or cell debris. From the microfiltration process a first permeate may be provided, said first permeate comprising RNA, DNA, vitamins, minerals and amino acids (free amino acids).

The first permeate obtained from the first separation process may be subjected to a second treatment separating the nucleic acids from the vitamins, minerals, and the amino acids (free amino acids).

In an embodiment of the present invention the second treatment involves a second membrane filtration, said second membrane filtration provides a second retentate comprising the nucleic acids and a second permeate comprising vitamins, minerals and/or amino acids; or a precipitation treatment where the nucleic acid is precipitated and a liquid fraction comprising vitamins, minerals and/or amino acids (free amino acids).

In another embodiment of the present invention, the second membrane filtration may be an ultrafiltration providing a second retentate comprising the nucleic acids and a second permeate comprising vitamins, minerals and/or amino acids (free amino acids).

Preferably, the second membrane filtration may have a molecular weight cut-off value (MWCO) in the range of 10,000-100,000 Dalton, such as in the range of 25,000-75,000 Dalton. Furthermore, it is preferred that the second membrane filtration may have a pore size in the range of 0.002-0.1 μm, such as a pore size in the range of 0.005-0.05 μm, e.g. a pore size in the range of 0.0075-0.01.

The second membrane filtration may involve an organic polymer membrane, such as polysulfones, poly (styrenes), PVDF (polyvinylidene fluoride) and PAN (polyacrylonitrile) including styrene-containing copolymers such as acrylonitrile-styrene, butadiene-styrene and styrene-vinylbenzylhalide copolymers, polycarbonates, cellulosic polymers, polypropylene, poly (vinyl chloride), poly (ethylene terephthalate); or an inorganic polymer membrane, such as a ceramic membrane filter material. Preferably, the second membrane filtration may involve an organic polymer membrane.

In an embodiment of the present invention, the ceramic membrane used in the first membrane filtration (and/or in the second membrane filtration) may be based on alumina, titanium, zirconia oxides, silicon carbide or some glassy materials.

In a further embodiment of the present invention, the second membrane filtration may be a dynamic disc filter.

In another embodiment of the present invention, the second treatment may involve precipitation of the nucleic acids. Preferably the nucleic acids may be precipitated from the vitamins, minerals, and amino acids by the addition of an organic alcohol, preferably an alcohol selected from ethanol or isopropanol.

Following the precipitation of the nucleic acids the organic alcohol, such as isopropanol or ethanol, may be removed from the supernatant by evaporation or distillation.

In a preferred embodiment of the present invention, the first permeate may be supplied to a second separation process which preferably involves an ultrafiltration process, preferably using ceramic membrane filter material; or a precipitation process where the nucleic acids are precipitated using ethanol or isopropanol. From the ultrafiltration process a second retentate may be provided, said second retentate comprising nucleic acids (RNA and DNA). From the ultrafiltration process a second permeate may be provided, said second permeate comprising vitamins, minerals and amino acids (free amino acids).

In an embodiment of the present invention, the ceramic membrane used in the first separation step; in the second separation step or in both separation steps, may be placed under pressure to improve capacity and/or effectivity of the membrane. Typically, a turbulent flow may be imparted to the biomass material in contact with the membrane and this turbulent flow agitates the liquids adjacent to the membrane and permits a higher content of solids in the retentate.

The pressure applied can be applied with a pump and/or with an inert gas under pressure to the biomass material.

Generally, products intended to enter the market as a food or a feed product or as an ingredient for consumption need to be treated to kill microbes (mainly bacteria) and eliminate pathogens. Pasteurization is one process often used in the food, feed and beverage industry to reduce the number of viable pathogens, so they are unlikely to cause disease.

In an embodiment of the present invention the one or more fraction or the SCP product may be pasteurized.

In the event the content of nucleic acids in the one or more fraction or in the SCP product needs to be even further reduced additional sequences of the one of, or the combination of, the separation processes, e.g. the membrane filtrations (microfiltration and ultrafiltration), as described herein may be run. Alternatively, additional removal of nucleic acids from the one or more fraction or the SCP product may involve enzymatic treatment of the one or more fraction or the SCP product.

In an embodiment of the present invention, the method further comprises the step
(vi) subjecting the first fraction or the first retentate obtained in step (iii); and/or the fourth fraction or the second permeate comprising vitamins, minerals and/or amino acids obtained in step (iv); and/or the fifth fraction or the SCP product provided in step (v) to an enzymatic treatment hydrolysing the remaining nucleic acids or fragments hereof to individual nucleotides.

The enzymes used for the hydrolysing of the remaining nucleic acids or fragments hereof to individual nucleotides may be selected from a nuclease, nucleosidase or a nucleotidase.

When enzymes have been added to reduce the nucleic acid content the enzymatic activity may be preferably inactivated. Hence, the method may further comprise the step
(vii) inactivation of the enzyme added in step (vi).

In an embodiment to of the present invention the process of cell disruption in step (ii); the first separation process in step (iii); the second treatment in step (iv); the preparation of the SCP product in step (v); the enzyme treatment in step (vi) and/or the enzyme inactivation in step (vii) is/are performed under controlled temperature conditions, preferably at a temperature of less than 50° C., particularly preferably from 25 to 50° C., e.g. from 25 to 35° C.

The method according to the present invention may furthermore, comprise the step of adding one or more unsaturated fatty acids, such as ARA, DHA and/or EPA, to the one or more fraction obtained from the present invention, such as the first retentate obtained in step (iii), to the second permeate obtained in step (iv) and/or to the SCP product obtained in step (v). Hence, in an embodiment of the present invention the one or more fraction, the first retentate, the second permeate, and/or the SCP product may be combined with one or more unsaturated fatty acids, such as ARA, DHA and/or EPA, preferably the SCP product may be combined with DHA.

The method of the present invention results in one or more unique fraction(s), or SCP product with several improved functionalities, reduced disadvantages and applications.

A preferred embodiment of the present invention relates to one or more fraction or a SCP product comprising a biomass material and a reduced content of nucleic acids, relative to the naturally occurring amount of nucleic acids in the biomass material.

It may be preferred that enzymatic degradation of the nucleic acids is not used on too large amount of nucleic acids, since the process simply results in the degradation of the nucleic acids to nucleotides and nucleosides but does not remove the components and the challenge with joints and kidneys, gout or gouty arthritis and kidney stones may still occur.

In an embodiment of the present invention the one or more fraction or the SCP product may comprise less than 90 mg nucleic acids per gram biomass material on a dry-matter basis, such as less than 75 mg/g biomass material, e.g. less than 50 mg/g biomass material, such as less than 25 mg/g biomass material, e.g. less than 1 mg/g biomass material, such as less than 750 μg/g biomass material, e.g. less than 500 μg/g biomass material, such as less than 100 μg/g biomass material, e.g. less than 10 μg/g biomass material.

It may be preferred that the one or more fraction or the SCP product may comprise a nucleic acid content of from 0.01 to 4.5% by weight on a dry-matter basis, such as 0.1 to 4%, e.g. from 1 to 3.5%, such as from 2 to 3%, e.g. about 2.2% by weight on a dry-matter basis.

In a further embodiment of the present invention the one or more fraction or SCP product may comprise a single-cell protein material. Furthermore, preferably, the one or more fraction or the SCP product comprises a methanotrophic bacteria.

As nucleic acids have been removed from the SCP product according to the present invention, the protein content of the SCP product of the present invention may be higher than prior art products where the nucleic acids are simply kept in the SCP product or where only enzymatic degradation of the nucleic acids has been introduced.

The one or more fraction or the SCP product according to the present invention may comprise at least 50% protein on a dry-matter basis, such as at least 60% protein on a dry-matter basis, e.g. at least 70% protein on a dry-matter basis, such as at least 80% protein on a dry-matter basis, e.g. at least 90% protein on a dry-matter basis, such as at least 95% protein on a dry-matter basis, e.g. in the range of 50-95% protein on a dry-matter basis, such as in the range of 60-85% protein on a dry-matter basis, e.g. in the range of 65-75% protein on a dry-matter basis, such as in the range of 68-83% protein on a dry-matter basis.

In an embodiment of the present invention, the one or more fraction or SCP product may be supplemented with one or more fatty acids. Preferably, the one or more fraction or SCP product may comprise one or more unsaturated fatty acids, such as ARA, DHA and/or EPA, preferably, the one or more fraction or SCP product may comprise DHA. The content of unsaturated fatty acids in the one or more fraction or SCP product may be dependent on the intended use of the product. In embodiment of the present invention the one or more fraction or SCP product comprises 0.5-15% (w/w) on a dry-matter basis of the unsaturated fatty acids.

Although the one or more fraction or SCP product according to the present invention may be used directly in, or as an ingredient for, food or feed products, the one or more fraction or SCP product may usually be further processed e.g. to remove excess water from the product. During the further processing the one or more fraction or SCP product may also be subjected to an additional drying step to provide a dry product comprising one or more fraction or SCP. The dry one or more fraction or SCP product may have a moisture content of 15% or less, such as 10% or less, e.g. 8% or less, such as 5% or less. The additional drying step may be provided by using a spray dryer.

The one or more fraction or SCP product according to the present invention may be used directly as a food or a feed product; or it may be used as an ingredient for a food or a feed product. In a preferred embodiment of the present invention the feed may be a fish feed or animal feed or human feed, preferably a fish feed or an animal feed.

In an embodiment of the present invention the one or more fraction or the SCP product may be used for food or feed or to be delivered in order to improve or optimize the health, performance or well-being of humans or animals, such as, but not limited to cloven hoofed animals (e.g. cattle, goats, sheep, pigs, etc.), poultry (e.g. fowls, chicken, ducks, goose/geese, turkey, etc.), fish (e.g. salmon, halibut, trout, cod, or other species bred in captivity) or shellfish (e.g. molluscs such as mussels, oysters, shrimps, prawns, lobsters, or scallops).

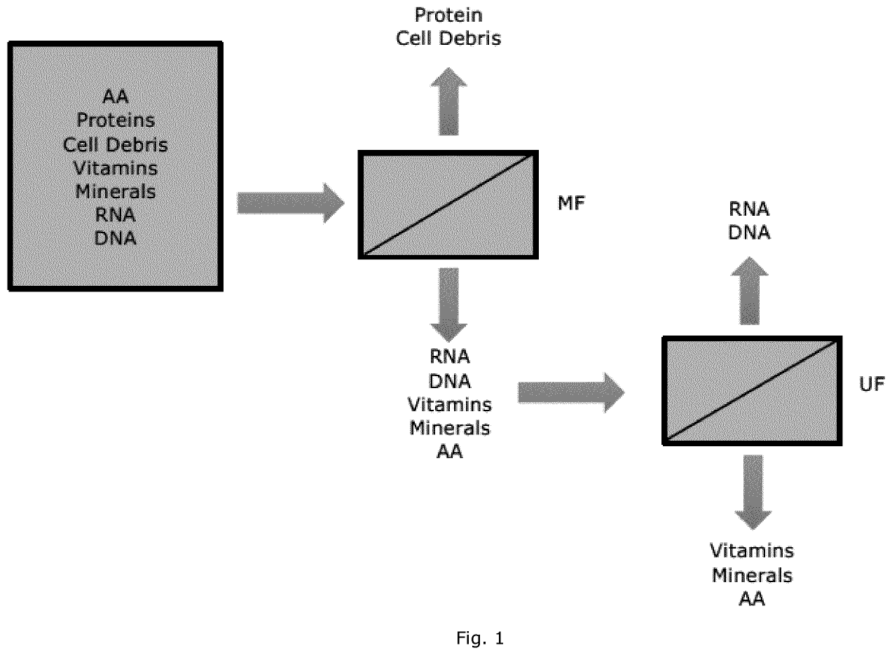

FIG. 1 illustrates an embodiment of the present invention where we are starting with providing the biomass material, comprising amino acids; proteins; cell debris; vitamins; minerals; RNA and DNA.

The biomass material may preferably be derived from a single cell protein material, particularly comprising methanotrophic bacteria. The preferred strain of methanotrophic bacteria being Methylococcus capsulatus (NCIMB 11132) is provided from NCIMB (National Collection of Industrial, Food and Marine Bacteria, Aberdeen, Scotland). As M. capsulatus can only oxidize hydrocarbons into the corresponding alcohols, aldehydes and carboxylic acids, but cannot oxidize higher hydrocarbons completely to carbon dioxide and water or utilize them for biomass production three other strains Alcaligenes acidovorans (NCIMB 13287), Bacillus firmus (NCIMB 13289) and Aneurinibacillus danicus (NCIMB 13288) are also provided and used together with M. capsulatus. The carbon source preferred by the present invention may be natural gas, biogas, syngas, methane or methanol.

After end fermentation, preferably in a continuous culture, the biomass material is harvested, this is not shown in FIG. 1, but the procedure of cultivating the biomass material and harvesting the biomass material is well known to the skilled person. Following the harvesting, part of the water is separated from the biomass material using an initial ultrafiltration process, increasing the dry-matter content from about 2% to about 15% (w/w). This initial ultrafiltration has a size exclusion in the range of about 100,000 Daltons, and the initial ultrafiltration process is performed at a temperature between 40 and 50° C.

The biomass material is then homogenised, and this process involves a change in pressure provided as a pressure drop 600-1500 bar leading to a disrupted biomass material. This process of homogenisation is not illustrated in FIG. 1, but is performed after harvesting of the biomass material and before applying the biomass material to the first separation process, the membrane filtration (MF).

The provided disrupted biomass material is subjected to MF using a semi-permeable dynamic disc filter with a pore size of 0.5 μm with 3 discoidal ceramic membranes. This device includes a backflush system that sends a part of the permeate back to the membrane every 20 sec. In this system, the rotating discs limit the membrane clogging and the formation of a polarisation layer. The MF is performed on a maximal period of 24 hours e.g. for a period less than 24 hours, e.g. for a period less than 15 hours, e.g. for a period less than 11 hours, e.g. for a period less than 8 hours, e.g. for a period less than 6 hours, e.g. for a period less than 4 hours.

The resulting first retentate obtained from the MF process comprises proteins and cell debris and the first permeate comprises the DNA, RNA and vitamins, minerals and amino acids.

The first permeate is then applied to a second membrane filtration being an ultrafiltration (UF). The UF membrane is a semi-permeable dynamic disc filter with a pore size of 20 nm or 5000 Da MWCO. This device also includes a backflush system that sends a part of the permeate back to the membrane every 20 sec. In this system, the rotating discs limit the membrane clogging and the formation of a polarisation layer. The UF is performed on a maximal period of 24 hours e.g. for a period less than 24 hours, e.g. for a period less than 15 hours, e.g. for a period less than 11 hours, e.g. for a period less than 8 hours, e.g. for a period less than 6 hours, e.g. for a period less than 4 hours.

The resulting second retentate obtained from the UF process comprises DNA and RNA, whereas the second permeate comprises the vitamins, minerals and amino acids.

The liquid component of the biomass material passes through the semi-permeable ceramic MF membrane, hereafter mentioned as first permeate. The component that does not pass through the semi-permeable MF ceramic membrane, hereafter mentioned as the first retentate has a higher concentration of cell debris and proteins, than the first permeate. The MF first retentate is collected, and the first permeate is continued for further separation by contacting the first permeate with the semi-permeable UF ceramic membrane under the foregoing conditions until the desired second retentate composition is obtained. The second permeate is the filtration liquid that passes through the semi-permeable UF ceramic membrane, hereafter mentioned as the second permeate. The component that does not pass through the semi-permeable UF ceramic membrane, hereafter mentioned as the second retentate has a higher concentration of nucleic acids, than the second permeate, the first permeate and the first retentate.

The one or more fraction or SCP product may be provided by combining the retentate of the MF (the first retentate) with the permeate of the UF (the second permeate). This combination results in a fermentation product having a reduced nucleic acid content relative to the biomass material and to the corresponding products described in the prior art.

Hence, the combined biomass fraction or SCP product, comprising the first retentate and a second permeate of two consecutive filtrations, provides a biomass fraction or a SCP product enriched in cell wall debris, proteins, minerals, vitamins and amino acids, obtained from processing the fermentation biomass, preferably, comprising methanotrophic bacteria.

In the present context, the terms “dry matter” and “ash” content was determined according to the A.O.A.C. method (reference A.O.A.C. Standard, 1945).

The DNA and total RNA concentration of the one or more fraction where assessed by phenol-chloroform extractions and nucleic acid concentration measurements, by measuring the absorbance at 260 nm. A phenol-chloroform extraction is a liquid-liquid extraction. A liquid-liquid extraction is a method that separates mixtures of molecules based on the differential solubility of the individual molecules in two different immiscible liquids. Liquid-liquid extractions are widely used to isolate DNA and total RNA (Agency for Toxic Substances and Disease Registry. Toxicological Profile for Chloroform. Atlanta, Ga.: U.S. Department of Health and Human Services, Public Health Service; 1997).

As an alternative to the use of ceramic membranes, use of organic polymer membranes with the appropriate adaptations, in either the first separation process and/or in the second separation process, can be used as well. Preferred organic polymers may be polysulfones, poly (styrenes), PVDF (polyvinylidene fluoride) and PAN (polyacrylonitrile) including styrene-containing copolymers such as acrylonitrile-styrene, butadiene-styrene and styrene-vinylbenzylhalide copolymers, polycarbonates, cellulosic polymers, polypropylene, poly (vinyl chloride), poly (ethylene terephthalate).

Even the above-mentioned sequence of steps is preferred, the vitamins, minerals and amino acids may be removed from the biomass material before the nucleic acid is removed from the proteins/peptides and/or the cell debris. In this case, the method according to the present invention, for removing nucleic acids from a biomass material comprises the steps of:
(i) providing the biomass material;
(ii) subjecting the biomass material to a process of cell disruption, providing a disrupted biomass material;
(iii) applying the disrupted biomass material to a first separation process resulting in a first retentate comprising proteins, cell debris and/or nucleic acid; and a first permeate comprising vitamins, minerals and/or amino acids;
(iva) suspending the first retentate in a liquid;
(ivb) subjecting the suspended first permeate to a second treatment separating the nucleic acids from proteins and/or cell debris;
(v) optionally, combining the first permeate obtained in step (iii) with the proteins and/or cell debris obtained in step (iv), providing a fraction wherein the nucleic acids have been removed.

Preferably, the liquid is an aqueous phase. The aqueous phase may preferably be water.

It should be noted that embodiments and features described in the context of one of the aspects of the present invention also apply to the other aspects of the invention.

All patent and non-patent references cited in the present application, are hereby incorporated by reference in their entirety.

### REFERENCES

WO 2010/069313;

WO 2000/70014;

US 2004/0241790

1-22. (canceled)

23. A method for providing one or more fraction(s) from a biomass material, the method comprising:
(i) providing the biomass material;
(ii) subjecting the biomass material to a process of cell disruption, providing a disrupted biomass material;
(iii) applying the disrupted biomass material to a first separation process resulting in a first fraction (first retentate) comprising proteins, and a second fraction (first permeate) comprising nucleic acids; and
(iv) subjecting the second fraction (first permeate) to a second treatment resulting in a third fraction (second retentate) comprising nucleic acid and a fourth fraction second permeate) comprising vitamins, minerals and/or amino acids.

24. The method according to claim 23, wherein the first fraction obtained in (iii) is combined with the fourth fraction obtained in (iv), providing a fifth fraction.

25. The method according to claim 23, wherein the first separation process comprises a first membrane filtration or a first chromatographic separation process.

26. The method according to claim 25, wherein the first membrane filtration is a microfiltration.

27. The method according to claim 23, wherein the second treatment involves a second membrane filtration or a second chromatographic separation process or a precipitation treatment where the nucleic acids are precipitated and a liquid fraction comprising vitamins, minerals and/or amino acids wherein said second membrane filtration provides a second retentate comprising the nucleic acids and a second permeate comprising vitamins, minerals and/or amino acids, and wherein the second membrane filtration is an ultrafiltration.

28. The method according to claim 23, wherein the biomass material is a single-cell protein material.

29. The method according to claim 23, wherein the biomass material comprises a methanotrophic bacteria.

30. A biomass fraction obtainable by a method according to claim 23, wherein the biomass fraction comprises at least 50% protein on a dry-matter basis.

31. A feed comprising the biomass fraction according to claim 30, preferably the feed is fish feed or animal feed or human food.

32. A method for providing a single cell protein product (SCP product) from a biomass material wherein said SCP product comprising a reduced amount of nucleic acids relative to the naturally occurring amount of nucleic acids in the biomass material, the method comprising:
(i) providing the biomass material;
(ii) subjecting the biomass material to a process of cell disruption, providing a disrupted biomass material;
(iii) applying the disrupted biomass material to a first separation process resulting in a first retentate comprising proteins and/or cell debris, and a first permeate comprising nucleic acids;
(iv) subjecting the first permeate to a second treatment separating the nucleic acids from vitamins, minerals and/or amino acids;
(v) optionally, combining the first retentate obtained in (iii) with the vitamins, minerals and/or amino acids obtained in (iv), providing the SCP product comprising a reduced amount of nucleic acids relative to the naturally occurring amount of nucleic acids.

33. The method according to claim 32, wherein the first separation process is a first membrane filtration.

34. The method according to claim 33, wherein the first membrane filtration is a microfiltration.

35. The method according to claim 32, wherein the second treatment involves a second membrane filtration or a second chromatographic separation process or a precipitation treatment where the nucleic acids are precipitated and a liquid fraction comprising vitamins, minerals and/or amino acids wherein said second membrane filtration provides a second retentate comprising the nucleic acids and a second permeate comprising vitamins, minerals and/or amino acids, and wherein the second membrane filtration is an ultrafiltration.

36. The method according to claim 32, wherein the biomass material is a single-cell protein material comprising a methanotrophic bacteria.

37. A biomass fraction obtainable by a method according to claim 32, wherein the biomass fraction comprises at least 50% protein on a dry-matter basis.

38. A feed comprising the biomass fraction according to claim 37, preferably the feed is fish feed or animal feed or human food.

39. A method for removing nucleic acids from a biomass material, the method comprises:
(i) providing the biomass material;
(ii) subjecting the biomass material to a process of cell disruption, providing a disrupted biomass material;
(iii) applying the disrupted biomass material to a first separation process resulting in a first retentate comprising proteins and/or cell debris, and a first permeate comprising nucleic acids;
(iv) subjecting the first permeate to a second treatment separating the nucleic acids from vitamins, minerals and/or amino acids;
(v) optionally, combining the first retentate obtained in (iii) with the vitamins, minerals and/or amino acids obtained in (iv), providing a SCP product wherein the nucleic acids have been removed.

40. The method according to claim 39, wherein the first separation process is a first membrane filtration.

41. The method according to claim 39, wherein the first membrane filtration is a microfiltration.

42. The method according to claim 39, wherein the second treatment involves a second membrane filtration or a second chromatographic separation process or a precipitation treatment where the nucleic acids are precipitated and a liquid fraction comprising vitamins, minerals and/or amino acids wherein said second membrane filtration provides a second retentate comprising the nucleic acids and a second permeate comprising vitamins, minerals and/or amino acids, and wherein the second membrane filtration is an ultrafiltration.

43. The method according to claim 39, wherein the biomass material is a single-cell protein material.

44. The method according to claim 39, wherein the biomass material comprises a methanotrophic bacteria.

45. A biomass fraction obtainable by a method according to claim 17, wherein the biomass fraction comprises at least 50% protein on a dry-matter basis.

46. A feed comprising the biomass fraction according to claim 45, preferably the feed is fish feed or animal feed or human food.
